(19) United States
(12) Patent Application Publication
(10) Pub. No.: US 2025/0345133 A1
(43) Pub. Date: Nov. 13, 2025

(54) MULTIFUNCTIONAL FERROMAGNETIC FIBER ROBOTS

(71) Applicant: VIRGINIA TECH INTELLECTUAL PROPERTIES, INC., Blacksburg, VA (US)

(72) Inventors: Xiaoting Jia, Blacksburg, VA (US); Yujing Zhang, Blacksburg, VA (US)

(21) Appl. No.: 18/854,347

(22) PCT Filed: Mar. 10, 2023
(86) PCT No.: PCT/US2023/064104

§ 371 Date: Oct. 4, 2024

Related U.S. Application Data
(60) Provisional application No. 63/364,535, filed on May 11, 2022.

(51) Int. Cl. A61B 34/30; A61B 1/00; G16H 40/67

(52) U.S. Cl. CPC A61B 34/30; A61B 1/00158; A61B 1/00165; G16H 40/67; A61B 2034/301

(57) ABSTRACT

Various embodiments of a multifunctional ferromagnetic fiber robot (MFFR) are described. According to one embodiment, the MFFR includes a central core and a ferromagnetic layer around the central core. The central core can include a waveguide, an electrode, and a hollow channel in one example. The ferromagnetic layer can include magnetic microparticles distributed in a thermoplastic elastomer. The waveguide can include silica or polymer waveguides. The electrode can include high-melting-point or low-melting-point metal electrodes. The MFFR includes or exhibits magnetic actuation properties that are activated in response to an external magnetic field. The magnetic actuation properties are adjustable based on a cross-sectional geometry of the central core and a particle loading concentration of the magnetic microparticles.

### CROSS REFERENCE TO RELATED APPLICATIONS

This application claims the benefit of and priority to U.S. Provisional Application Ser. No. 63/364,535, filed May 11, 2022, titled “MULTIFUNCTIONAL FERROMAGNETIC FIBER ROBOTS,” the entire contents of which are hereby incorporated herein by reference.

### STATEMENT OF FEDERALLY SPONSORED RESEARCH OR DEVELOPMENT

This invention was made with government support under Grant No. CAREER 1847436 awarded by the National Science Foundation (NSF). The government has certain rights in the invention.

### BACKGROUND

Small-scale robotic devices have great potential for use in the biomedical and other fields. Over the past few decades, small-scale robotic devices have seen breakthroughs in design and application. These devices have the potential to access hard-to-reach regions in human bodies and perform various medical procedures in a minimally invasive manner. Additionally, small-scale robotic devices may be used for biomechanics characterization and drug delivery. These devices have the ability to operate in confined spaces and delicate environments and even be remotely controlled in some cases.

### SUMMARY

Various embodiments of a multifunctional ferromagnetic fiber robot (MFFR) are described. In one embodiment, the MFFR includes a central core and a ferromagnetic layer around the central core. The central core can include a waveguide, an electrode, and a hollow channel. The ferromagnetic layer can include magnetic microparticles distributed in a thermoplastic elastomer. The magnetic microparticles can include neodymium magnet particles according to one example. The thermoplastic elastomer (TPE) can include styrene-ethylene-butylene-styrene (SEBS) according to one example, but other TPE materials may be selected for the MFFR.

The MFFR includes or exhibits magnetic actuation properties, which are activated in response to an external magnetic field. The magnetic actuation properties are adjustable based on a cross-sectional geometry of the central core and a particle loading concentration of the magnetic microparticles in the thermoplastic elastomer. The electrode can be embodied as low-melting point metal electrodes or high-melting-point metal electrodes. The low-melting-point metal electrodes can include a Tin-Bismuth (BiSn) electrode and the high-melting-point metal electrodes can include a Silver (Ag) electrode according to one example.

The waveguide can be embodied as a silica waveguide or a step-index polymer waveguide composed of a polycarbonate core and a polymethyl methacrylate (PMMA) cladding according to one example. The waveguide can be centrally located in the central core. The electrode can be positioned at one side of the waveguided in the central core, and the hollow channel can be positioned at another side of the waveguide in the central core according to one example. The MFFR can be configured to deflect toward a direction of a magnetic field that is applied perpendicularly to the MFFR.

In another embodiment, the MFFR includes a central core and a ferromagnetic layer around the central core. The central core can include a waveguide and a hollow channel, where the waveguide is centrally located in the central core, and the hollow channel is distributed around the waveguide. The ferromagnetic layer can include magnetic microparticles distributed in a thermoplastic elastomer. The MFFR includes or exhibits magnetic actuation properties. The magnetic actuation properties are activated in response to an external magnetic field, where the magnetic actuation properties are adjustable based on a cross-sectional geometry of the central core and a particle loading concentration of the magnetic microparticles in the thermoplastic elastomer. The MFFR can be configured to deflect toward a direction of the external magnetic field being applied perpendicularly to the MFFR.

An electrode may be omitted or added to the central core. If an electrode is added, the electrode can include low-melting-point electrodes or high-melting-point metal electrodes, such as a tin-bismuth (BiSn) electrode or a silver (Ag) electrode. The electrode may be positioned in the central core around the waveguide. The waveguide can be embodied as a silica waveguide or a step-index polymer waveguide composed of a polycarbonate core and a polymethyl methacrylate (PMMA) cladding.

In another embodiment, the MFFR can include a central core and a ferromagnetic layer around the central core. The central core can include an electrode and a hollow channel. The ferromagnetic layer can include magnetic microparticles distributed in a thermoplastic elastomer. The MFFR includes magnetic actuation properties. The magnetic actuation properties are activated in response to an external magnetic field, where the magnetic actuation properties are adjustable based on a cross-sectional geometry of the central core and a particle loading concentration of the magnetic microparticles in the thermoplastic elastomer. The MFFR can be configured to deflect toward a direction of the external magnetic field being applied perpendicularly to the MFFR.

A waveguide may be omitted or added to the central core. If a waveguide is added, the waveguide can be located centrally in the central core, and the electrode and the hollow channel can be distributed around the waveguide.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates a preform that can be used for fabricating a multifunctional ferromagnetic fiber robot (MFFR) in accordance with various embodiments of the present disclosure.

FIG. 2 illustrates a second preform that can be used for fabricating another MFFR in accordance with various embodiments of the present disclosure.

FIG. 3 illustrates a third preform that can be used for fabricating another MFFR in accordance with various embodiments of the present disclosure.

FIG. 4 illustrates a preform preparation process for the preform shown in FIG. 1 in accordance with various embodiments of the present disclosure.

FIG. 5 illustrates a cross-sectional view of a fiber drawn from the preform shown in FIG. 1 in accordance with various embodiments of the present disclosure.

FIG. 6 illustrates a cross-sectional view of a fiber drawn from the preform shown in FIG. 2 in accordance with various embodiments of the present disclosure.

FIG. 7 illustrates a cross-sectional view of a fiber drawn from the preform shown in FIG. 3 in accordance with various embodiments of the present disclosure.

FIG. 8 illustrates the magnetization loop of a neodymium iron boron (NdFeB) composite used in ferromagnetic layers shown in FIGS. 5-7.

FIG. 9 illustrates a comparison of the remanent magnetization of the NdFeB composite before and after the thermal drawing process in accordance with various embodiments of the present disclosure.

FIG. 10 illustrates a schematic of an MFFR fabricated from one of the fibers illustrated in FIGS. 5-7, deflecting towards the direction of the uniform magnetic field B being applied perpendicularly to the MFFR in accordance with various embodiments of the present disclosure.

FIG. 11 illustrates a calculated effective Young's modulus of an MFFR plotted against the core-to-fiber ratio in accordance with various embodiments of the present disclosure.

FIG. 12 illustrates normalized deflection δ/L predicted from theory and experimental measurements plotted against the applied field strength with different core-to-fiber ratios in accordance with various embodiments of the present disclosure.

FIG. 13 illustrates normalized deflections δ/L predicted from theory and experimental measurements plotted against core-to-fiber ratios under different magnetic field strengths in accordance with various embodiments of the present disclosure.

FIG. 14 illustrates an exemplary MFFR in accordance with various embodiments of the present disclosure.

### DETAILED DESCRIPTION

Small-scale robotic devices that are capable of remotely navigating through complex and dynamic environments are promising for biomedical applications. Owing to their flexibility and steerability, these robotic devices can potentially offer minimally invasive, localized, and targeted diagnostic and therapeutic procedures for next-generation percutaneous coronary intervention (PCI), atrial fibrillation (AF) ablation, gastrointestinal endoscopy, brain surgery, and other procedures where operating space is confined.

Despite the great potential of biomedical robotic devices, several challenges exist today which hinder their applicability to the clinical setting. For example, there are difficulties in scaling robotic devices down to the micrometer scale, which limits the types of lesions that can be accessed. Further, inefficiency and inaccuracy of the guidance and navigation process can also impede the delivery of localized and precise therapy deep inside the body, and the lack of integrated multimodal sensing and treatment systems can restrict the functions that can be achieved via robotic devices.

To address some of these challenges, researchers have developed ferromagnetic soft robots composed of flexible polymer matrices with doped ferromagnetic microparticles. The response of the ferromagnetic soft robots to external magnetic fields can be precisely predicted and designed by calculating the generated torques or forces using quantitative models. As the actuation relies on the dispersed ferromagnetic microparticles, these robotic devices can be miniaturized and encoded on a microscale, which makes them a promising approach for minimally invasive surgery.

Despite the advantages offered by the ferromagnetic soft robots, a major challenge in these devices is the lack of multimodal diagnostic and therapeutic functions. Recently, several attempts have been made to enable multimodal capabilities in microscale robotics. Conventional clean-room technology allows for the fabrication of micro-robotic probes with integrated electronic components for heating and flow sensing. However, the total length of these devices is limited by the size of the silicon wafer, which is well below the length requirement for most interventional surgeries. In addition, the fabrication process involves complicated and costly procedures. Injection molding can be used to incorporate simple components such as an optical fiber or a hollow channel into robots, but realizing complex multi-material structures with a micro-scale resolution remains difficult. So far, scalable submillimeter ferromagnetic robots with multiple diagnostic and therapeutic functions have yet to be developed.

According to various embodiments, multifunctional ferromagnetic fiber robots (MFFRs) that address the problems discussed above are presented and described herein. The MFFRs can be fabricated using a thermal drawing process (TDP) or a convergence drawing process involving a fiber preform with integrated electrical, optical, and microfluidic components. The MFFRs are fabricated from a fiber drawn from the preform and can include some or all of the electrical, optical, and microfluidic components that are integrated in the preform and can be magnetically actuated or steered in response to an external magnetic field. Depending on the desired use case of the MFFR (e.g., PCI, AF ablation, gastrointestinal endoscopy, brain surgery, etc.), different arrangements of the integrated electrical, optical, and microfluidic components can be integrated into the preform, which can be transferred to the MFFR during the thermal drawing process.

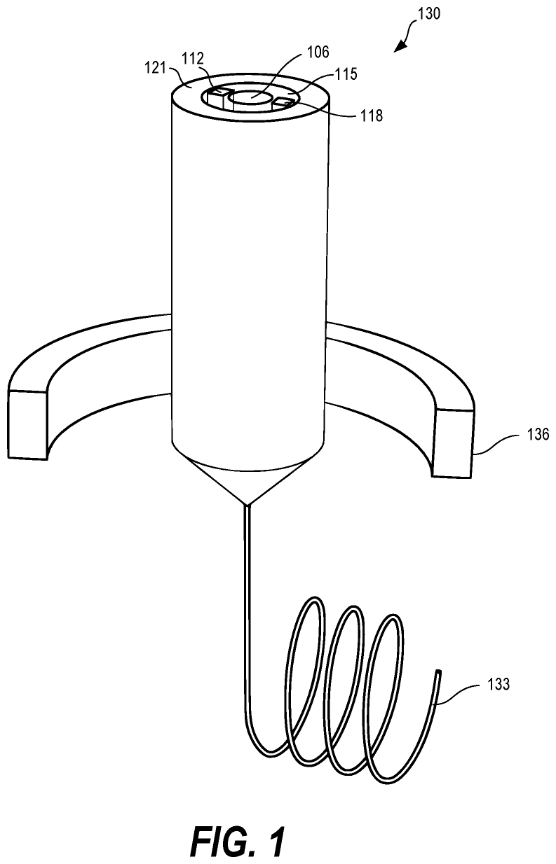

Turning now to the drawings, FIG. 1 illustrates a preform 130 (“preform 130”) that can be used for fabricating MFFRs in accordance with various embodiments of the present disclosure. In the example shown, the preform 130 can integrate a functional core 115 (“core 115”) with various components, such as waveguides, microfluidic channels, and electrodes, among other various components. The integrated materials and components allow for magnetically controlled steering, optical signal delivery and collection, fluid delivery, and electrical stimulation and recording for the MFFRs, among possibly additional functions or features. The core 115 can also provide the mechanical support required for probe insertion during minimally invasive surgeries according to some examples.

The core 115 is centrally located in the preform 130 and can include various electrical, optical, and microfluidic components. In one example, the core 115 includes a centrally located waveguide 106, a hollow channel 118 located at one side of the waveguide 106, and an electrode 112 located at another side of the waveguide 106. The waveguide 106 can be embedded into the core 115 and be embodied as step-index polymer waveguides made of polycarbonate core and polymethyl methacrylate (PMMA) cladding, commercially available PMMA waveguides, silica waveguides, and other types of waveguides. In one example, the polycarbonate core can have a coefficient value of n=1.58, while the PMMA cladding can have a coefficient value of n=1.49. The electrode 112 can be embodied as a low-melting-point metal electrode, such as a tin-bismuth (BiSn) electrode, or a high-melting-point metal electrode, such as a silver (Ag) electrode. Other low-melting-point and high-melting-point metal electrodes may be relied upon in some cases.

In some embodiments, the arrangement of the electrode 112 and the hollow channel 118 in the core 115 can vary as compared to what is depicted. For example, the electrode 112 can be positioned at a same side of the waveguide 106 as the side the hollow channel 118 is positioned instead of being positioned at different sides. In another example, the core 115 may include more than one hollow channel, more than one electrode or more than one hollow channel and more than one electrode. The arrangement and quantity of electrodes and hollow channels in the core 115 may be determined based on the desired use case of the MFFR (e.g., PCI, AF ablation, gastrointestinal endoscopy, brain surgery, etc.).

The core 115 is surrounded by a ferromagnetic layer 121, which can include embedded ferromagnetic microparticles. The ferromagnetic layer 121 can include ferromagnetic composite (FC) layers composed of varying thicknesses. In one example, the ferromagnetic layer 121 can be prepared by dispersing neodymium, iron, and boron (NdFeB) particles in thermoplastic elastomers (TPEs) using a hot press. The NdFeB particles can be evenly dispersed in the TPEs, to the extent possible, in some cases. The TPE materials can include thermoplastic styrenic block copolymers (SBCs), thermoplastic elastomer polyolefins (TPOs), thermoplastic vulcanizates (TPVs), thermoplastic polyurethanes (TPUs), thermoplastic copolyester (TPC), thermoplastic polyamides (TPAs), and non-classified thermoplastic elastomers (TPZs), among other related and suitable materials. According to a representative example, styrene-ethylene-butylene-styrene (SEBS) was selected as the TPE material for the ferromagnetic layer 121 due to its low elastic modulus, good biocompatibility, and compatibility with TDP techniques.

According to one example, the NdFEB composite can be prepared by thermally mixing SEBS (G1657, Kraton™) and NdFeB microparticles with an average diameter of 5 um (MQFP-B+, Magnequench). SEBS pellets can be pressed into sheets at 180° C. using a hot press and the sheets can be weighted. The NdFEB particles can be weighted for desired volume percentage loading and then sprinkled between the SEBS sheets. Next, the SEBS sheets with deposited NdFEB can be pressed in a hot press at 180° C. and 50 bar for 10 minutes to embed the NdFeB particles into the SEBS sheets. To make a homogeneous dispersion of NdFEB particles in SEBS, the obtained sheets can be folded and pressed at the same conditions for 8-10 cycles.

Once formed, the preform 130 can be heated with a heater 136 above the glass-transition temperature of the integrated polymers in the preform 130 and pulled into an approximately 150-m-long fiber at a speed of about 4 m/min under an applied external stress and high temperature of about 260-300° C. to produce fiber 133. The fiber 133 can be drawn from a drawing tower and has the same cross-sectional geometry and composition as the preform 130 but with a 20-150-fold reduction in dimension (i.e., circumferential dimension). A loading concentration of 35% (v/v) was applied in one example, which is the greatest concentration achieved when drawing at the above temperatures. With higher loading concentrations, the viscosity of the FC composite layers can become too high, which makes the layers unsuitable for the thermal drawing process.

Alternatively, materials with high melting temperatures, such as silver wires and silica waveguides, can also be integrated into the MFFR via a convergence drawing process. Silver wires or silica waveguides can be threaded into the channels inside the preform 130 and converged with the surrounding polymer during the pull down procedure. The fiber 133 can be drawn to a length greater or shorter than 150-m in some cases. Various MFFRs can be fabricated from the fiber 133 once drawn, and the magnetic properties of the ferromagnetic layer 121 enable the MFFRs to be remotely controlled based on magnetic fields that may be generated and controlled by a user, as described below.

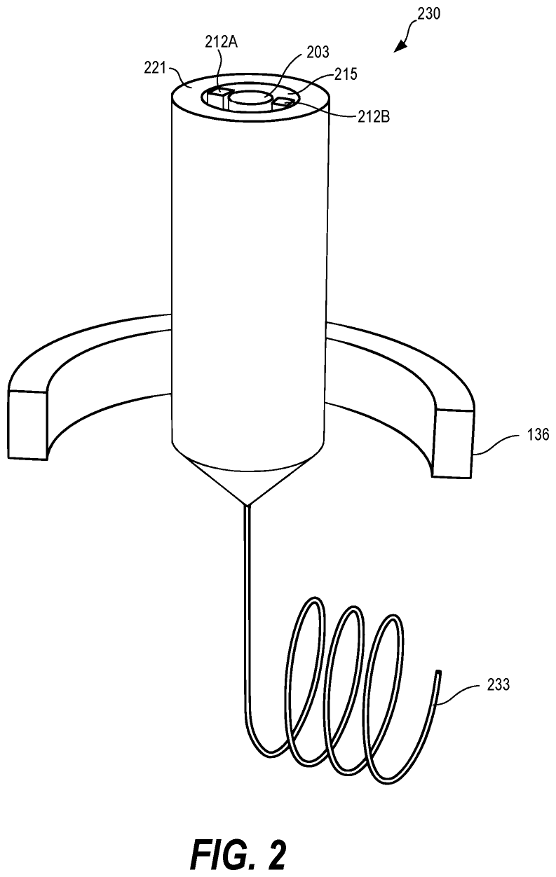

FIG. 2 illustrates a preform 230 that can be used for fabricating MFFRs in accordance with another embodiment of the present disclosure. The preform 230 can be used in a way similar to that of the preform 130 to fabricate the MFFRs, but with a convergence drawing process. The preform 230 integrates a functional core 215 (“core 215”), similar to the integration of the core 115 for the preform 130. The core 215 is centrally located in the preform 230 and can include various electrical, optical, and microfluidic components. The core 215 includes a hollow channel 203 and a pair of electrodes 212A and 212B distributed around the hollow channel 203. The core 215 is surrounded by a ferromagnetic layer 221, which can include FC layers doped with ferromagnetic microparticles, similar to that of the ferromagnetic layer 121.)

Similar to that of the ferromagnetic layer 121, the ferromagnetic layer 221 can include FC layers of varying thicknesses. The ferromagnetic layer 221 can be prepared by dispersing NdFeB particles in TPE materials using a hot press. The TPE materials can include thermoplastic SBCs, TPOs, TPVs, TPUs, TPCs, TPAs, and non-classified TPZs. According to a representative example, SEBS was selected as the TPE material for the ferromagnetic layer 221 due to its low elastic modulus, good biocompatibility, and compatibility with the TDP techniques. The NdFEB composite can be prepared similarly to the way discussed above in connection with the ferromagnetic layer 121 of the preform 130.

The preform 230 is suitable for fabricating MFFRs with integrated components with high melting temperatures, such Ag wires and silica waveguides. Accordingly, the electrodes 212A and 212B can be embodied as high-melting-point metal electrodes, such as Ag electrodes or wires. Silver wires can be threaded into the channels inside the preform 230 and converged with the surrounding polymer during the pulling down procedure.

The preform 230 can be heated in a way similar to how the preform 130 is heated, and fiber 233 may be pulled into an approximately 150-m-long fiber at about 4 m/min under an applied external stress and high temperature of about 260-300° C. The fiber 233 can be drawn from the preform 230 from a drawing tower and can have the same cross-sectional geometry and composition as the preform 230 but with a 20-150-fold reduction in dimension (i.e., circumferential dimension). A loading concentration of 35% (v/v) was applied in one example, which is the greatest concentration achieved when drawing at the above temperatures. With higher loading concentrations, the viscosity of the FC composite layers can become too high, which makes the layers unsuitable for the thermal drawing process.

The fiber 233 can be drawn to a length greater or shorter than 150-m in some cases similar to that of the fiber 133. Various MFFRs can be fabricated from the fiber 233 once drawn, and the magnetic properties of the ferromagnetic layer 221 enable the MFFRs to be remotely controlled based on magnetic fields that may be generated and controlled by a user. In some cases, the core 215 may include more than two electrodes, based on the desired use case of the MFFR (e.g., PCI, AF ablation, gastrointestinal endoscopy, brain surgery, etc.).

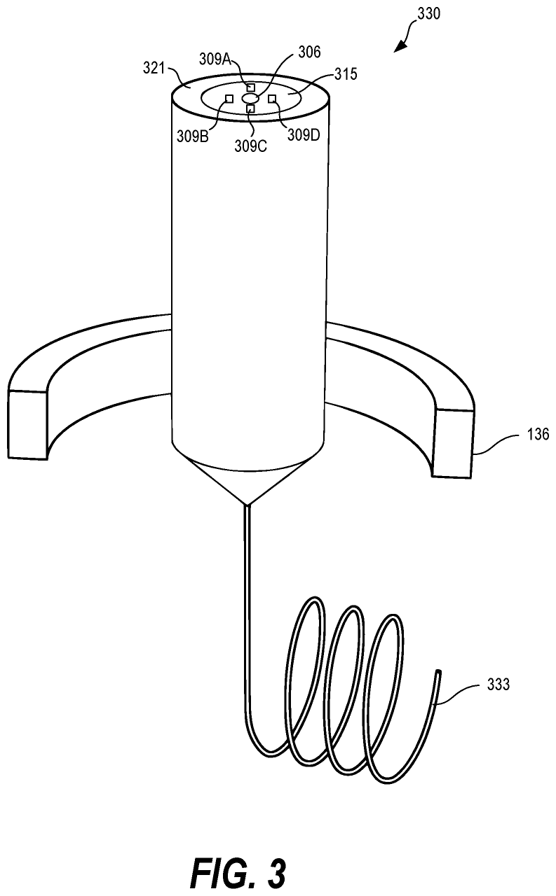

FIG. 3 illustrates a preform 330 that can be used for fabricating MFFRs in accordance with another embodiment of the present disclosure. The preform 330 can be used in a way similar to that of the preforms 130 and 230 to fabricate the MFFRs, with a convergence drawing process. The preform 330 integrates a functional core 315 (“core 315”), similar to the integration of the core 115 for the preform 130 and the core 215 for the preform 230. The core 315 is centrally located in the preform 230 and can include various electrical, optical, and microfluidic components. The core 315 includes a waveguide 306 located centrally in the core 315 and a plurality of hollow channels 309A-309D located around the waveguide 306. The core 315 is surrounded by a ferromagnetic layer 321, which can include ferromagnetic composite (FC) layers doped with ferromagnetic microparticles, similar to that of the ferromagnetic layers 121 and 221.

Similar to that of the ferromagnetic layers 121 and 221, the ferromagnetic layer 321 can include FC layers of varying thicknesses. The ferromagnetic layer 321 can be prepared by dispersing NdFeB particles in TPEs using a hot press. The TPE materials can include thermoplastic SBCs, TPOs, TPVs, TPUs, TPCs, TPAs, and non-classified TPZs. According to a representative example, SEBS was selected as the TPE material for the ferromagnetic layer 321 due to its low elastic modulus, good biocompatibility, and compatibility with the TDP techniques. The NdFeB composite can be prepared similarly to the way discussed above in connection with the ferromagnetic layers 121 and 221 of the preforms 130 and 230, respectively.

The preform 330 is suitable for fabricating MFFRs with integrated components with low or high melting temperatures. The waveguide 306 is similar to the waveguide 106 in that it can be embedded centrally in the core 115 and include step-index polymer waveguides made of polycarbonate core and PMMA cladding, commercially available PMMA waveguides, silica waveguides, and other types of waveguides. In one example, the polycarbonate core can have a coefficient value of n=1.58, while the PMMA cladding can have a coefficient value of n=1.49.

The preform 330 can be heated in a way similar to how the preform 130 and the preform 230 are heated, and the fiber 333 may be pulled into an approximately 150-m-long fiber at about 4 m/min under an applied external stress and high temperature of about 260-300° C. The fiber 333 can be drawn from the preform 330 from a drawing tower and can have the same cross-sectional geometry and composition as the preform 330 but with a 20-150-fold reduction in dimensions (i.e., circumferential dimension). A loading concentration of 35% (v/v) was applied in one example, which is the greatest concentration achieved when drawing at the above temperatures. With higher loading concentrations, the viscosity of the FC composite layers can become too high, which makes the layers unsuitable for the thermal drawing process.

The fiber 333 can be drawn to a length greater or shorter than 150-m in some cases similar to that of the fibers 133 and 233. Various MFFRs can be fabricated from the fiber 333 once drawn, and the magnetic properties of the ferromagnetic layer 321 enable the MFFRs to be remotely controlled based on magnetic fields that may be generated and controlled by a user. In some cases, the core 315 may include fewer or greater than three hollow channels, based on the desired use case of the MFFR (e.g., PCI, AF ablation, gastrointestinal endoscopy, brain surgery, etc.).

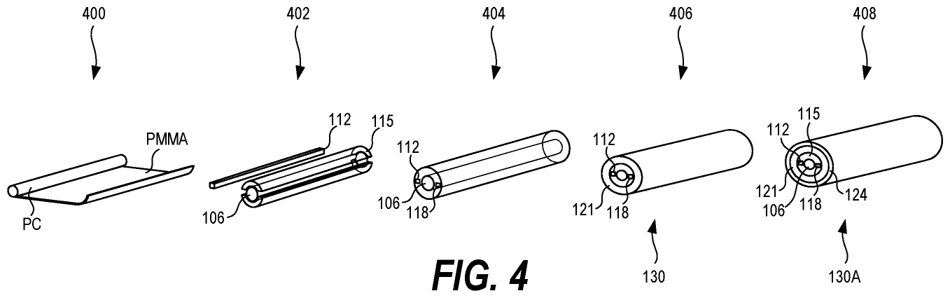

FIG. 4 illustrates a preparation process for the preform 130 in accordance with various embodiments of the present disclosure. The process is provided as a representative example and, in some cases, can vary as compared to that shown. At step 400, the preparation process includes adding or wrapping a relatively thin cladding layer of PMMA around a polycarbonate (PC) core rod. The process also includes adding a thicker layer of PC over the PC/PMMA core and consolidating the components at 180° C. in a vacuum oven. The PC rod, PMMA cladding layer, and outer PC layer correspond to the components of the waveguide 106, for example, as a step-index polymer waveguide made of polycarbonate core and PMMA cladding. The outer, thicker layer of polycarbonate corresponds to the surrounding material in the functional core 115.

Next, at step 402, the process includes forming or machining two grooves in the outer PC layer (i.e., in the surrounding material of the functional core 115) of the components consolidated at step 400. The grooves can be formed at any suitable locations around the waveguide 106. In the example shown in FIG. 4, the grooves are formed at opposite sides of the waveguide 106, but the grooves can be formed at other locations. In some cases, more than two grooves can be formed. At step 404, the process includes filling one of the grooves by the electrode 112, while the other groove is retained for the hollow channel 118.

Next, at step 406, the process includes wrapping a ferromagnetic layer 121 around the rod formed in step 402. The ferromagnetic layer 121 can include one, two, or more layers of material. In one example, the ferromagnetic layer 121 can be embodied as an inner thinner layer of PC, a thicker layer of SEBS doped with NdFeB particles, and another outer layer of PC. The components shown at step 406 of FIG. 4 are consolidated again in a vacuum oven to form the preform 130. The preforms 230 and 330 can be prepared using similar process steps but with implementation of different electrical, optical, and microfluidic components as can be appreciated.

In some cases, the preparation process may include, at step 408, wrapping a separate sacrificial outer layer around the components shown in step 406, as a support for the thermal drawing process. For example, the preparation process can involve wrapping an outer layer 124 around the ferromagnetic layer 121. The outer layer 124 can include a polycarbonate or PMMA layer. The outer layer 124 can be consolidated in a vacuum, along with the other layers, to form the preform 130A. After the thermal drawing process, the outer layer 124 may be etched away from the resulting fiber (e.g., from the fiber 133) using acetone with the assistance of ultrasonic.

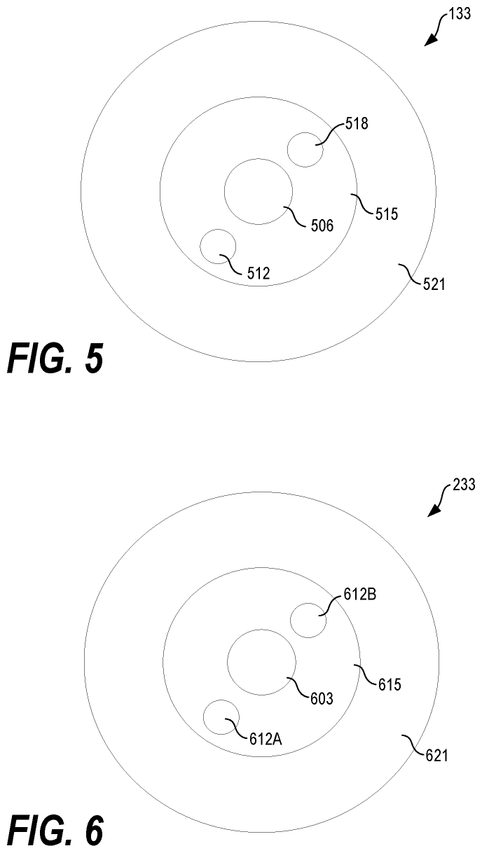

FIG. 5 illustrates a cross sectional view of the fiber 133 fabricated with a thermal drawing process from the preform 130 according to an embodiment of the present disclosure. As mentioned above, the fiber 133 includes the various ferromagnetic, electrical, optical, and microfluidic components found in the preform 130 but with a 20-150-fold reduction in dimensions. For instance, the fiber 133 includes a functional core 515 (“core 515”) commensurate with the properties of the core 115, with a reduction in dimensions. The fiber 133 also includes an electrode 512 and a hollow channel 518 in the core 515. Similarly, the electrode 512 is commensurate with the properties of the electrode 112, and the hollow channel 518 is commensurate with the properties of the hollow channel 118, with a reduction in dimensions. The fiber 133 also includes a waveguide 506, which is commensurate with the properties of the waveguide 106, with a reduction in dimensions. The core 515 is surrounded by a ferromagnetic layer 521, which is commensurate with the properties of the ferromagnetic layer 121, but with a reduction in dimensions.

In one embodiment, the ferromagnetic layer 521 includes a ferromagnetic composite (FC), and the electrode 512 includes a BiSn electrode. Various MFFRs can be fabricated from the fiber 133, and variants of the FC or the BiSn electrode may be implemented for the ferromagnetic layer 521 and the electrode 512 depending on the desired use case of the MFFR (e.g., PCI, AF ablation, gastrointestinal endoscopy, brain surgery, etc.). If variants are to be implemented, such as a silver electrode implementation rather than a BiSn electrode implementation, the preform 130 should be integrated first with the variant components, to transfer the variant components to the fiber 133 during the drawing process.

FIG. 6 illustrates a cross sectional view of the fiber 233 fabricated with a thermal drawing process involving the preform 230 according to an embodiment of the present disclosure. As mentioned above, the fiber 233 can integrate the various ferromagnetic, electrical, optical, and microfluidic components found in the preform 230 but with a 20-150-fold reduction in dimensions. As discussed above in connection with FIG. 2, a waveguide may be omitted from the preform 230, and the preform 230 includes the electrodes 212 that are distributed around the hollow channel 203, both of which are positioned in the core 215.

Thus, the fiber 233 includes a functional core 615 (“core 615”) commensurate with the properties of the core 215, with a reduction in dimensions. The fiber 233 also includes electrodes 612A and 612B and a hollow channel 603 in the core 615. The electrodes 612A and 612B are commensurate with the properties of the electrodes 212A and 212B, and the hollow channel 603 is commensurate with the properties of the hollow channel 203, both with a reduction in dimensions. The core 615 is surrounded by a ferromagnetic layer 621, which is commensurate with the properties of the ferromagnetic layer 221, but with a reduction in dimensions.

In one exemplary embodiment, the ferromagnetic layer 621 includes FC layers, and the electrodes 612A and 612B include Ag electrodes. Various MFFRs can be fabricated from the fiber 233, and variants of the FC layers or the Ag electrodes may be implemented for the ferromagnetic layer 621 and the electrodes 612A and 612B depending on the desired use case of the MFFR (e.g., PCI, AF ablation, gastrointestinal endoscopy, brain surgery, etc.). If variants are to be implemented, such as BiSn electrodes rather than Ag electrodes, the preform 230 should be integrated first with the variant components, to transfer the variant components to the fiber 233 during the drawing process. The fiber 233 may be drawn based on a convergence drawing process.

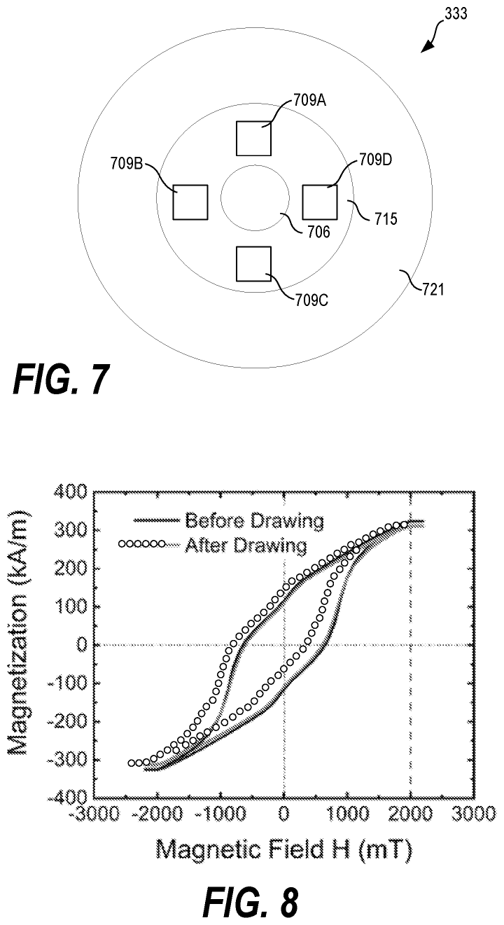

FIG. 7 illustrates a cross sectional view of the fiber 333 fabricated with a thermal drawing process involving the preform 330 according to an embodiment of the present disclosure. As mentioned above, the fiber 333 can integrate the various ferromagnetic, electrical, optical, and microfluidic components found in the preform 330 but with a 20-150-fold reduction in dimensions. As discussed above in connection with FIG. 3, electrodes may be omitted from the preform 330, and the preform 330 includes the hollow channels 309 that are distributed around the waveguide 306, both of which are positioned in the core 315.

Similarly, the fiber 333 includes a functional core 715 (“core 715”) commensurate with the properties of the core 315, with a reduction in dimensions. The fiber 333 also integrates hollow channels 709A-709D and a waveguide 706 in the core 715. The hollow channels 709A-709D are commensurate with the properties of the hollow channels 309, and the waveguide 706 is commensurate with the properties of the waveguide 306, both with a reduction in dimensions. The core 715 is surrounded by a ferromagnetic layer 721, which is commensurate with the properties of the ferromagnetic layer 721, but with a reduction in dimensions.

In one exemplary embodiment, the ferromagnetic layer 721 includes FC layers, and the waveguide 706 includes silica waveguide or a polymer waveguide. Various MFFRs can be fabricated from the fiber 333, and variants of the FC and the silica and polymer waveguide may be implemented for the ferromagnetic layer 721 and the waveguide 706 depending on the desired use case of the MFFR (e.g., PCI, AF ablation, gastrointestinal endoscopy, brain surgery, etc.). If variants are to be implemented, such as a silica waveguide implementation rather than a polymer waveguide implementation, the preform 330 should be integrated first with the variant components, to transfer the variant components to the fiber 333 during the drawing process. The fiber 333 may be drawn based on a thermal or a convergence drawing process.

After the thermal or convergence drawing processes described above, fiber tips of the fibers 133, 233, and 333 can be magnetized along the fiber axis with a magnetic field using a high-field electromagnet. In one example, the fiber tips were magnetized with a magnetic field of 2.2 T using a high-field electromagnet. It is worth noting that the high temperature applied during the drawing process may have only a modest effect on the magnetic properties of the NdFeB composite in the ferromagnetic layers 521, 621, and 721 of the fibers 133, 233, and 333.

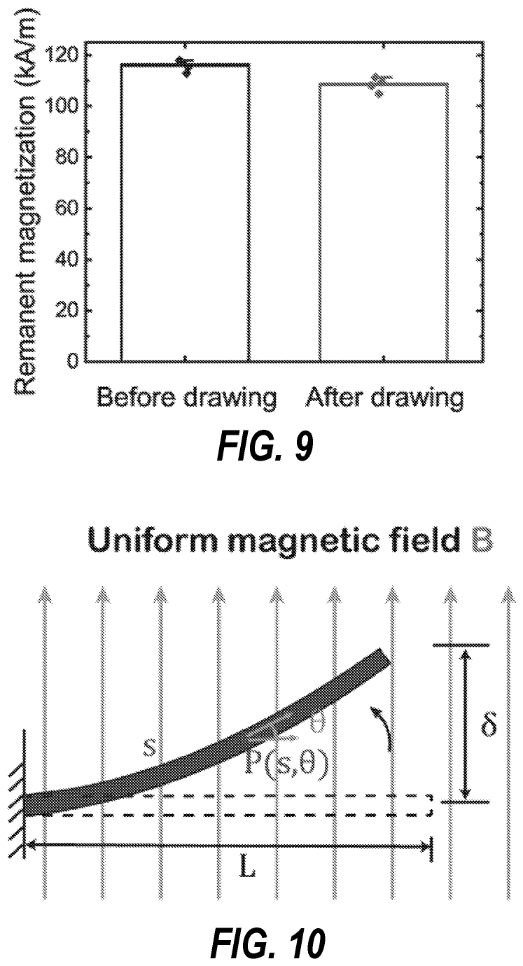

FIG. 8 illustrates the magnetization loop of the NdFEB composite (20% (v/v)) used in the ferromagnetic layers 521, 621, and 721 in accordance with various embodiments described herein. FIG. 8 indicates that the magnetization became saturated when the applied magnetic field strength reached 2 T. FIG. 9 illustrates a comparison of the remanent magnetization of the FC composite (NdFEB composite) before and after the thermal drawing process in accordance with various embodiments of the present disclosure. After the drawing process, the remanent magnetization of the composite dropped by around <10%, changing from 116±2 kA/m to 109±2 kA/m, as illustrated in FIG. 9.

The magnetic actuation properties of MFFRs fabricated from the fibers 133, 233, and 333 can depend on their magnetic and mechanical properties, both of which are affected by the fiber geometry and particle loading concentrations. The effects of these factors were analyzed based on a simplified model, where the fibers 133, 233, and 333 were considered as a beam with a length/and placed perpendicular to a uniform magnetic field, as illustrated in FIG. 10.

FIG. 10 illustrates a schematic of a MFFR fabricated from the fibers 133, 233, or 333, deflecting towards the direction of the uniform magnetic field B applied perpendicularly to a length or longitudinal axis of the MFFR in accordance with various embodiments described herein. The unconstrained length of the MFFR is denoted L. The symbol δ indicates the deflection of the free end of the MFFR, s denotes the arc length from the fixed point to the point of interest (denoted by P), and θ denotes the angle between the tangent to the curve at point P and the reference direction.

The governing equation describing the MFFR's response to a uniform magnetic field B can be expressed as

$$\frac{{E}_{e f f} I}{A} \frac{{d}^{2} θ}{{ds}^{2}} + {M}_{e f f} B cos θ = 0 , \qquad ( 1 )$$

where s denotes the arc length from the fixed point to the point of interest (denoted by P in FIG. 10), θ denotes the angle between the tangent to the curve at point P and the reference direction, E_{eff} is the effective Young's modulus of the whole beam, I is the area moment of inertia, which can be expressed as I=πD⁴/64 for a cylindrical beam, A is the cross-section area of the whole beam, which can be expressed as A=πD²/4, and M_{eff} is the effective magnetization density of the whole fiber, which can be described as

$${M}_{e f f} = {M}_{p} ϕ ( 1 - {( \frac{d}{D} )}^{2} ) , \qquad ( 2 )$$

where Mₚ denotes the magnetization density of the particles, and ϕ denotes the particle loading volume concentration. The effective Young's modulus of the MFFR can be calculated by

$${E}_{e f f} = {{E}_{c o r e} ( \frac{d}{D} )}^{4} + {E}_{j a cket} ( 1 - {( \frac{d}{D} )}^{4} ) , \qquad ( 3 )$$

where E_{core} and E_{jacket} denote the Young's moduli of the fiber core and ferromagnetic microparticles loaded SEBS. To simplify the model, a fiber structure that has a polycarbonate core can be used with the E_{core} being set as 2.4 GPa.

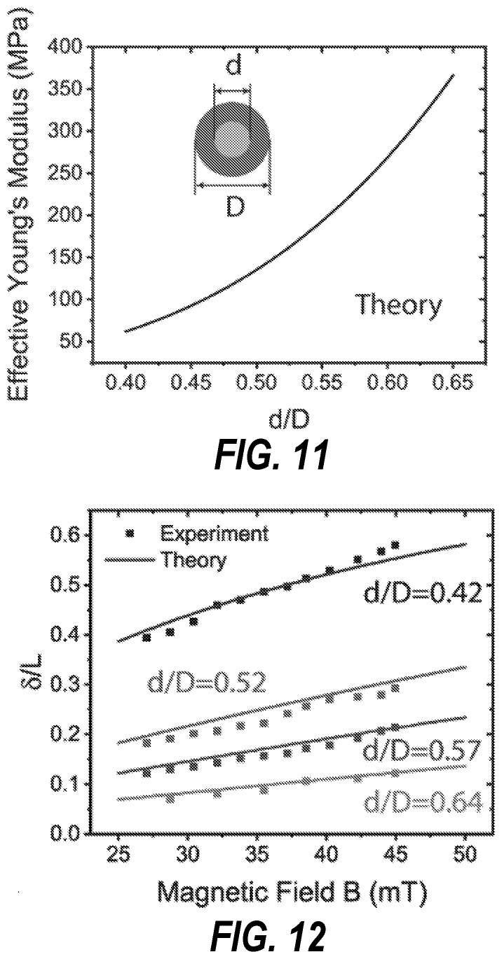

FIG. 11 illustrates a calculated effective Young's modulus of the MFFR plotted against the core-to-fiber ratio in accordance with various embodiments of the present disclosure. The fiber structure of the MFFR is simplified as a polycarbonate core (diameter, d) and an FC jacket (outer diameter, D, loading fraction, 35% (v/v)). FIG. 11 shows that the effective Young's modulus of the MFFR increases nonlinearly as the core-to-fiber ratio d/D increases when E_{jacket} is set as 11 MPa with a particle doping concentration of 35% (v/v). A d/D range of 0.4 to 0.65 was selected for further investigation. When d/D is too small, the fiber becomes too soft, and thus cannot provide the stiffness and mechanical strength required for the probe insertion process. On the other hand, a larger d/D results in a larger stiffness of the fiber, which limits its actuation response under a magnetic field.

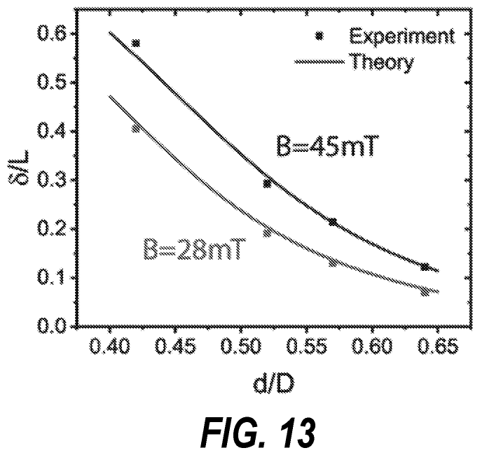

FIG. 12 illustrates normalized deflection δ/L predicted from theory and experimental measurements plotted against the applied field strength with different core-to-fiber ratios in accordance with various embodiments of the present disclosure. FIG. 13 illustrates normalized deflections δ/L predicted from theory and experimental measurements plotted against core-to-fiber ratios under different magnetic field strengths in accordance with various embodiments of the present disclosure.

By substituting Equations 2 and 3 into Equation 1, the deflection of the magnetically active fiber tip can be obtained. The predicted fiber deflection ratios δ/L from modeling show good agreement with experimental data shown in FIGS. 12 and 13 under various core-to-fiber ratios d/D and magnetic field strengths. The normalized deflection ratios δ/L of the fibers are 0.12, 0.21, 0.31, and 0.58 at 45 mT for different core-to-fiber ratios of d/D=0.42, 0.52 0.57, and 0.64 when L/D=40, respectively. When other conditions remain unchanged, a smaller core-to-fiber ratio can lead to a smaller effective Young's modulus and a larger effective magnetization density, resulting in a larger response to the same external magnetic field. Also, when the magnetic particles loading concentration ϕ and core-to-fiber ratio d/D are in the range of 0-35% (v/v) and 0.4-0.7, respectively, the fiber deflections can increase with the loading concentration. Based on this model, MFFRs can be designed by tuning their geometries and materials to fit the mechanical and magnetic actuation requirements for different application scenarios.

In addition, the Young's modulus of the doped SEBS can increase nonlinearly as the NdFEB particles increase, which can be described using the Mooney Equation with the assumption that the particles are closed packed spheres, and the composites are incompressible,

$${E}_{jacket} = {E}_{0} exp ( \frac{2.5 ϕ}{1 - 1.35 ϕ} ) , \qquad ( 4 )$$

where E₀ denotes the Young's modulus of undoped SEBS, which is 2.1 MPa. The governing equation can be simplified as

$$\frac{{E}_{e f f} I}{A} \frac{{d}^{2} θ}{{ds}^{2}} + {M}_{e f f} B cos θ = 0 , \qquad ( 5 )$$

where the angular displacement at the free end of the fiber θ_{L} can be obtained by solving

$$L = \sqrt{\frac{{E}_{e f f} I}{2 {M}_{e f f} B A}} {\int }_{0}^{{θ}_{L}} \frac{d θ}{\sqrt{sin {θ}_{L} - sin θ}} . \qquad ( 6 )$$

The deflection of the free end of the fiber along the y-axis is

$$δ = \sqrt{\frac{{E}_{e f f} I}{2 {M}_{e f f} B A}} {\int }_{0}^{{θ}_{L}} \frac{sin θ d θ}{\sqrt{sin {θ}_{L} - sin θ}} . \qquad ( 7 )$$

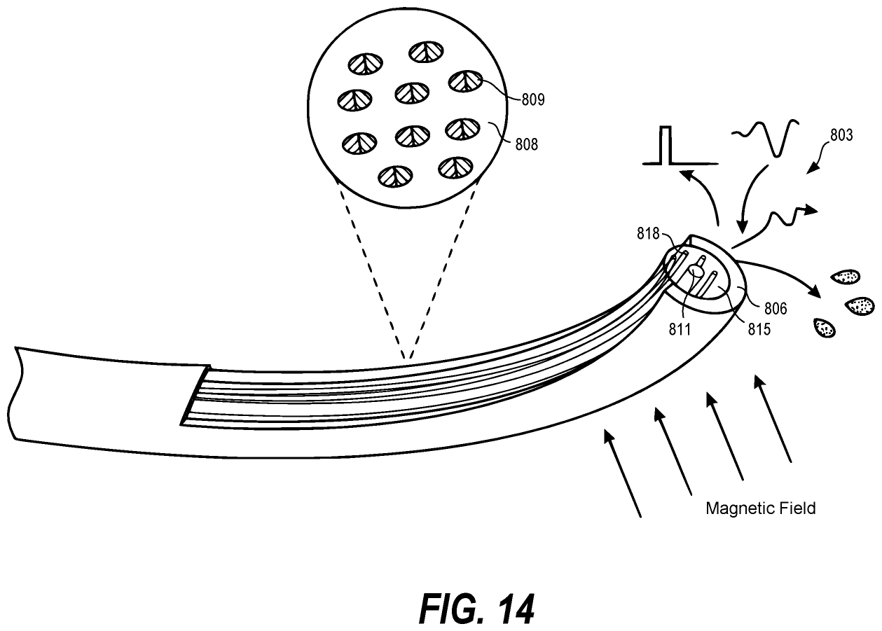

FIG. 14 illustrates an exemplary MFFR 803 in accordance with various embodiments of the present disclosure. The MFFR 803 can be fabricated from a preform similar to any of the preforms 130, 230, or 330 and can include segments of any of the fibers 133, 233, or 333, depending on the selected preform. The MFFR 803 includes a functional core 815 (“core 815”) that is surrounded by a ferromagnetic layer 806. Various optical, electrical, and microfluidic components may be integrated into the core 815.

The ferromagnetic layer 806 can be similar in material and properties with the ferromagnetic layers 521, 621, and 721. Similar to that of the ferromagnetic layers 521, 621, and 721, the ferromagnetic layer 806 can include FC layers of varying thicknesses. The FC layers can include a thermoplastic elastomer 808 and magnetic microparticles 809 that are doped in the thermoplastic elastomer 808. In one example, the FC layer can be prepared by evenly dispersing NdFeB particles as the magnetic microparticles 809 in TPEs using a hot press. However, other TPE materials can be used, such as thermoplastic SBCs, TPOs, TPVs, TPUs, TPCs, TPAs, and non-classified TPZs, among others.

According to a representative example, SEBS was selected as the TPE material for the thermoplastic elastomer 808 in the ferromagnetic layer 806 due to its low elastic modulus, good biocompatibility, and compatibility with the thermal drawing process (TDP). The NdFeB composite can be prepared similarly to the way discussed above in connection with the ferromagnetic layers 521, 621, and 721 of the preforms 130, 230, and 330, respectively.

The MFFR 803 can include a waveguide 811 and various electric or microfluidic components 818 integrated in the core 815. The microfluidic components 818 can be embodied as electrodes, hollow channels, or a combination of electrodes and hollow channels. Although four electric or microfluidic components 818 are depicted, fewer or greater than four components may be implemented in the core 815 depending on desired use case use case of the MFFR 803 (e.g., PCI, AF ablation, gastrointestinal endoscopy, brain surgery, etc.). Further, the components 818 can be embodied as having a combination of various electrodes and/or hollow channels. For example, the integrated components 818 can be embodied as four electrodes, two hollow channels and two electrodes, four hollow channels, etc.

The waveguide 811 is commensurate with the properties of the waveguides 506 and 706 (FIGS. 5 and 7), and the electrodes or the hollow channels of the integrated components 818 are commensurate with the properties of the electrodes 512 and 612 (FIGS. 5 and 6) and/or the hollow channels 518, 603, and 709 (FIGS. 5, 6, and 7). In some cases, the waveguide 811 maybe omitted from the MFFR 803.

The MFFR 803 has or exhibits magnetic actuation properties and steering capabilities that may be activated in response to an external magnetic field. The magnetic field may be user-controlled and be applied in a direction perpendicular to the axis of the MFFR 803. Based on the cross-sectional geometry of the core 815 and a particle loading concentration of the magnetic microparticles 809, the magnetic actuation properties of the MFFR 803 may be adjusted to meet the needs of a particular use case. The MFFR 803 can be configured to deflect toward the direction of the external magnetic field being applied to the MFFR 803.

The magnetic steering capabilities of the MFFR 803 may provide therapeutic functions in the clinical setting, such as light therapy and fluid delivery using the MFFR 803. Light and optical technologies are widely used in modern medicine for diagnosis, therapy, and surgery including angioplasty, photodynamic therapy, photothermal therapy, and light-triggered drug release. In addition, conventional catheters often contain tubes that enable localized delivery of contrast agents and embolization agents. According to one example, the MFFR 803 with one waveguide, four microfluidic channels, and an FC jacket was used to reach a model silicone vessel (modeled after a human vessel) with a first aneurysm under the guidance of an external magnetic field. After reaching the first aneurysm with a sharp turn at t=19 s, the MFFR 803 changed directions, passed the second aneurysm, and emitted a red laser light. Next, it turned to another branch, and injected red liquid dye into the third aneurysm. These results demonstrate that the MFFR 803 enables remote steering with larger angles compared with conventional guidewires owing to its active steering capability, and simultaneous therapeutic treatment including light delivery and drug injection.

The application of the MFFR 803 in interventional endocardia surgery using a human cardiac model was also demonstrated in another experiment. The MFFR 803 was inserted from the superior vena cava, and under the guidance of an external magnetic field (20 to 60 mT) generated by a permanent magnet at a distance (50 to 70 mm), the MFFR 803 reached the target site at the right ventricle near the apex. Then, red dye from a microfluidic channel inside the MFFR 803 was injected to indicate the location of the fiber tip. To further validate the diagnostic and therapeutic functions of the multifunctional fiber robots, EGM, pacing, and bioimpedance monitoring experiments were performed using a Langendorff-perfused mouse heart model.

To mimic the scenario in interventional cardiovascular surgeries, the MFFR 803 was inserted from the superior vena cava of a mouse heart. The MFFR 803 passed the right atrium and tricuspid valve and reached the right ventricle with the tip gently touching the inner wall close to the apex. A whole heart electrocardiogram (ECG) was recorded with three leads in the bath while localized bipolar EGM was recorded simultaneously through two exposed silver electrodes at the fiber tip, implying good contact between the fiber and the ventricle wall. The bipolar EGM reached the negative peak when the cardiomyocytes underneath the electrodes depolarized. This depolarization happened between the R peak (apex ventricle activation) and S peak (base ventricle activation) recorded in ECG, which also verified that the fiber tip was localized between the apex and base of the right ventricle. Next, electrical pulses were delivered through the two electrodes inside the MFFR 803 and successfully paced the heart as shown in the recorded ECG. Before pacing, the R-R duration was around 220 ms. Upon pacing, the heart was forced to follow the paces of the stimulating pulses with a cycle length of 150 ms. After pacing for about 20 s, the stimulating signal was stopped, and the heart gradually recovered to the original stage with a R-R reduction of around 220 ms.

Myocardial tissue impedance decreases with cardiac edema, which is associated with heart disease. Thus, it is important to monitor myocardial tissue impedance during cardiovascular surgeries, such as in off-pump coronary artery bypass (OPCAB) surgery. In order to investigate if the MFFR 803 could monitor myocardial tissue impedance in the presence of cardiac edema, two-electrode bioimpedance measurements were performed through electrodes inside the MFFR 803 before and after mannitol perfusion. To obtain a lower noise level and better chemical stability, the surface of the silver electrodes were chlorinated by immersing the MFFR 803 in FeCl₃ solution for 1 min. A scanning electron microscopy (SEM) image showed the sub-micron structures on the tip surface after chlorination, and the corresponding energy-dispersive X-ray spectroscopy verified the presence of the Ag/AgCl layer. From the measured impedance spectra, it was observed that the myocardial impedance dropped significantly after mannitol perfusion, while the perfusion solution impedance stayed consistent. Specifically, at 10 kHz, the myocardial impedance dropped from 35±15 kΩ to 15±5 k≤Ω (Welch's t-test, p-value: 0.003, n=9 measurements, n=3 hearts). This impedance drop is believed to be due to mannitol-induced extracellular edema.

Compared to conventional millimeter-scale catheters, the MFFR 803 with a submillimeter size and active steering capability offer additional benefits in precise control and localized sensing, especially for operation on small hearts. For instance, cardiac intervention for pediatric patients with congenital heart disease requires small devices for both diagnostic and therapeutic catheterization. Pre-clinical cardiac research is another potential application of the MFFR 803. The current electrophysiological research on isolated heart models is limited to the measurement of epicardial action potential and bioimpedance. The MFFR 803 can provide sensing and modulation tools for cardiac research involving endocardial measurement in the intact small heart. The MFFR 803 can potentially be improved to sense more physiological conditions, including temperature, blood pressure, blood oxygen, etc.

The MFFR 803 may also be used in conjunction with microscale electroporation and chemical delivery. Electroporation is the phenomenon of creating pores in the lipid bilayers of cell membranes when the applied potential differences are greater than the transmembrane voltage. These pores can be either reversible or irreversible, depending on their size and the number of pores formed. Specifically, reversible electroporation can be combined with drug delivery to treat disease or infection sites and increases the synergistic uptake of drugs. To evaluate the electroporation and drug delivery capability of the MFFR 803, U-251 glioblastoma cells were cultured in hydrogels in 3D-printed scaffolds and delivered reversible electroporation (RE) treatments to produce a volume of electroporated cells. Glioblastomas are the most commonly occurring cranial tumor, with a life expectancy under current treatment modalities averaging a little over a year after diagnosis. U-251 cells are between 20 to 40 μm in length and have an average area of 700 μm² in a 5 mg/mL collagen hydrogel.

The MFFR 803 with two Ag electrodes and one microfluidic channel was inserted into the gel. During testing, electric pulses were applied through the two electrodes while 5 mM of Ca²⁺ adjuvant was introduced through the microfluidic channel to reduce the electric field threshold (EFT). Pulse parameters were set to 200 pulses with a pulse width of 100 μs, pulse amplitude of 175 V, and a repetition rate of 1 Hz. To simulate the electric field distribution during electroporation, finite element analysis (FEA) was also performed of the experiment. Due to the minimal gap of 140 μm between the charged leads and the insulated walls of the conducting wires, the electric field decayed rapidly from the fiber surface. Simulations also showed no observable temperature increase over the entire treatment. For the in vitro reversible electroporation results, unaffected cells expressed red fluorescence while membrane-electroporated cells expressed green fluorescence due to the uptake of YO-PRO-1 molecules. The measured RE EFT was between 450-500 V/cm, higher than that reported in the literature. This may be due to the small number of cells present within the lesion, reducing the accuracy of lesion size. The negligible heating of the gel may be another reason, as temperature rise is known to reduce EFTs. These results show that effective localized reversible electroporation treatment and chemical delivery can be simultaneously achieved through the MFFR 803.

The MFFR 803 can also be used for in vivo neural electrophysiological recording and optogenetic control. Implantable neural probes that penetrate brain tissue can record various types of electrophysiological signals and perform neural modulation. However, it is challenging to target the probes to the desired location precisely, especially under complex conditions with obstacles, such as significant fiber tracts, tumors and vessels. These complications can be significantly minimized by using steerable and flexible devices, such as magnetic needles, which can reach deep brain region using curved trajectories and bypass critical structures. The MFFR 803 is sufficiently miniaturized to produce a steerable multifunctional neural probe with an overall diameter of 0.3 mm. With the magnetically steerable jackets and the assistance of external magnetic fields, the neural probes can be inserted in a more controllable and precise manner by avoiding obstacles, allowing access to some out-of-reach places, and reducing neurovascular damages. Without an external magnetic field, the probe was straightly implanted in a brain phantom (0.6 wt % agarose gel). With the control of a permanent magnet, the probe can be implanted with a deflected angle of about 45°. Red dye can then be injected through the inner microfluidic channel to indicate the location of the probe tip. The impedance of the fiber probes at 1 kHz was found to be 271+46 KΩ, making them suitable for spike recording.

To evaluate the functionality of the fiber probes in recording localized brain activities, the fiber probes were implanted into the hippocampal region of wild-type mice (n=4). The embedded electrode captured spiking activities from multiple neurons, from which was found two distinctly insulated clusters via principal component analysis (PCA). The quality of the isolation was evaluated by L-ratios and isolation distance, which were 0.05 and 75, respectively. The two clusters were also observed in the raw trace and spiking traces as marked with the corresponding matched colors in PCA, which demonstrates the fiber probes can record neural activities with a single-unit resolution. The power spectral density analysis of the recorded trace showed that brain oscillations were observed in the frequency range of 6-10 Hz, corresponding to the theta oscillations in the hippocampal network pattern of activity in mice.

In addition, it was demonstrated that simultaneous optical modulation and electrophysiological readout can be achieved using the MFFR 803. Transmission spectroscopy confirmed the utility of the probes for optical guidance in the visible range (450-750 nm). Specifically, the transmission attenuation measured using the cut-back method was 0.797 dB/cm at a wavelength of 473 nm, which was the excitation peak of channelrhodopsin2 (ChR2). During the experiment, the waveguide inside the MFFR 803 was coupled to a silica optical fiber using a direct ferrule-to-ferrule coupling, while the electrode was electrically connected to a pin. Next, the fiber probes were implanted in Thy1-ChR2-YFP mice, which express ChR2 across the nervous system, in the hippocampal region (n=4). Laser pulses were applied at a frequency of 10 Hz with a pulse width of 5 ms and a power density of 5.1 mW/mm² through the fiber probes, and recorded optically evoked neural electrophysiological activities using the electrodes inside the fiber probes. These results demonstrate that the MFFR 803 can be magnetically steered in a brain phantom, record single-unit electrophysiological signals, and perform optogenetic stimulation and recording in mice.

The MFFR 803 may also be used as a fiber bundle endoscope. Optical fiber bundle endoscopes are widely used for imaging, sensing, and illumination in hard-to-reach locations of the human body. With the MFFR 803, fiber bundle endoscopes can be produced with active steering in a single step. The MFFR 803 can include a fiber bundle core for imaging and a ferromagnetic jacket for steering. PMMA fibers were chosen for imaging pixels due to their flexibility and low melting temperature. The diameter of the imaging core was about 350 μm with 320 pixels. The size of each pixel was about 20 μm and the overall diameter of the fiber being about 600 μm. The number of pixels and imaging resolution can be further improved by using smaller fibers for bundling in the preform and increasing the draw-down-ratio during the TDP. The transmission spectrum shows that this thermally drawn imaging fiber could guide light across the visible range and the attenuation can be ˜0.316 dB/cm at a wavelength of 615 nm.

For the experimentation performed, a halogen broadband light was used that is delivered by a multimode fiber, and the light was focused onto a custom-made mask. Then the pattern on the mask was imaged onto the distal end of the imaging fiber. During the experiment, the pattern focused on the fiber end was enlarged by 1.2 times compared to the original mask after the lens set. Images were collected from the other end of the fiber through a camera. For demonstration, two different patterns (a 100-μm-thick line and a character “C”) were successfully captured via a 40-cm-long imaging fiber with distinguishable images.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The features, structures, and components described herein may be combined in one or more embodiments in any suitable manner, and the features discussed in the various embodiments are interchangeable in many cases. Terms such as “a,” “an,” “the,” and “said” are used to indicate the presence of one or more elements and components. The terms “comprise,” “include,” “have,” “contain,” and their variants are used to be open ended and may include or encompass additional elements, components, etc., in addition to the listed elements, components, etc., unless otherwise specified. The terms “first,” “second,” etc. are used only as labels, rather than a limitation for a number of the objects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A fiber robot, comprising:
a central core, the central core comprising a waveguide, an electrode, and a hollow channel; and
a ferromagnetic layer around the central core.

2. The fiber robot of claim 1, wherein the ferromagnetic layer comprises magnetic microparticles distributed in a thermoplastic elastomer.

3. The fiber robot of claim 2, wherein the magnetic microparticles comprise neodymium magnet particles.

4. The fiber robot of claim 2, wherein the thermoplastic elastomer comprises styrene-ethylene-butylene-styrene (SEBS).

5. The fiber robot of claim 2, wherein the fiber robot exhibits magnetic actuation properties, the magnetic actuation properties being activated in response to an external magnetic field, the magnetic actuation properties being adjustable based on a cross-sectional geometry of the central core and a particle loading concentration of the magnetic microparticles in the thermoplastic elastomer.

6. The fiber robot of claim 1, wherein the electrode comprises low-melting-point metal electrodes or high-melting-point metal electrodes, the low-melting-point metal electrodes comprising a Tin-Bismuth (BiSn) electrode and the high-melting-point metal electrodes comprising a Silver (Ag) electrode.

7. The fiber robot of claim 1, wherein the waveguide comprises a silica waveguide or a step-index polymer waveguide composed of a polycarbonate core and a polymethyl methacrylate (PMMA) cladding.

8. The fiber robot of claim 1, wherein the waveguide is centrally located in the central core.

9. The fiber robot of claim 1, wherein:
the electrode is positioned at one side of the waveguide in the central core; and
the hollow channel is positioned at another side of the waveguide in the central core.

10. The fiber robot of claim 1, wherein the fiber robot is configured to deflect toward a direction of a magnetic field being applied perpendicularly to the fiber robot.

11. A fiber robot, comprising:
a central core, the central core comprising a waveguide and a hollow channel, the waveguide being located centrally in the central core and the hollow channel being distributed around the waveguide; and
a ferromagnetic layer around the central core, the ferromagnetic layer comprising magnetic microparticles distributed in a thermoplastic elastomer, wherein:
the fiber robot exhibits magnetic actuation properties, the magnetic actuation properties being activated in response to an external magnetic field, the magnetic actuation properties being adjustable based on a cross-sectional geometry of the central core and a particle loading concentration of the magnetic microparticles in the thermoplastic elastomer.

12. The fiber robot of claim 11, wherein the magnetic microparticles comprise neodymium magnet particles.

13. The fiber robot of claim 11, wherein the thermoplastic elastomer comprises styrene-ethylene-butylene-styrene (SEBS).

14. The fiber robot of claim 11, wherein:
the central core comprises an electrode:
the electrode comprises low-melting-point metal electrodes or high-melting-point metal electrodes: and
the low-melting-point metal electrodes comprise a tin-bismuth (BiSn) electrode and the high-melting-point metal electrodes comprise a silver (Ag) electrode.

15. The fiber robot of claim 14, wherein the electrode is positioned in the central core around the waveguide.

16. The fiber robot of claim 11, wherein the waveguide comprises a silica waveguide or a step-index polymer waveguide composed of a polycarbonate core and a polymethyl methacrylate (PMMA) cladding.

17. The fiber robot of claim 11, wherein the fiber robot is configured to deflect toward a direction of the external magnetic field, the external magnetic field being applied perpendicularly to the fiber robot.

18. A fiber robot, comprising:
a central core, the central core comprising an electrode and a hollow channel; and
a ferromagnetic layer around the central core, the ferromagnetic layer comprising magnetic microparticles distributed in a thermoplastic elastomer, wherein:
the fiber robot exhibits magnetic actuation properties, the magnetic actuation properties being activated in response to an external magnetic field, the magnetic actuation properties being adjustable based on a cross-sectional geometry of the central core and a particle loading concentration of the magnetic microparticles in the thermoplastic elastomer; and
the fiber robot is configured to deflect toward a direction of the external magnetic field.

19. The fiber robot of claim 18, wherein:
the magnetic microparticles comprise neodymium magnet particles; and
the thermoplastic elastomer comprises styrene-ethylene-butylene-styrene (SEBS).

20. The fiber robot of claim 18, further comprising a waveguide, wherein:
the waveguide is located centrally in the central core; and
the electrode and the hollow channel are distributed around the waveguide.
